(19) United States
(12) Patent Application Publication
(10) Pub. No.: US 2026/0355186 A1
(43) Pub. Date: Nov. 12, 2026

(54) PHARMACEUTICAL COMPOSITIONS COMPRISING ACALABRUTINIB MALEATE

(71) Applicant: NATCO PHARMA LIMITED, Hyderabad (IN)

(72) Inventors: Krishna Murthy BHAVANASI, Hyderabad (IN); Satyanarayana VATTIKUTI, Hyderabad (IN); Naveen Krishna YALAMANCHALLI, Hyderabad (IN); Pavan BHAT, Hyderabad (IN); Venkaiah Chowdary NANNAPANENI, Hyderabad (IN)

(21) Appl. No.: 19/121,621

(22) PCT Filed: Oct. 11, 2023
(86) PCT No.: PCT/IN2023/050921

§ 371 Date: Apr. 16, 2025

(30) Foreign Application Priority Data
Oct. 17, 2022 (IN) 202241059139

(51) Int. Cl. A61K 31/4985; A61K 9/20; A61K 9/28

(52) U.S. Cl. CPC A61K 31/4985; A61K 9/2013; A61K 9/2018; A61K 9/2027; A61K 9/2054; A61K 9/2059; A61K 9/2095; A61K 9/2893

(57) ABSTRACT

The present invention relates to pharmaceutical compositions comprising a Bruton tyrosine kinase (BTK) inhibitor. More particularly, the present invention relates to a composition comprising Acalabrutinib maleate and one or more pharmaceutically acceptable excipients and process for preparing such compositions.

### FIELD OF THE INVENTION

The present invention relates to pharmaceutical compositions comprising a Bruton tyrosine kinase (BTK) inhibitor. More particularly, the present invention relates to a composition comprising Acalabrutinib maleate and one or more pharmaceutically acceptable excipients and process for preparing such compositions.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Acalabrutinib is an inhibitor of Bruton's tyrosine kinase (BTK). The chemical name for Acalabrutinib is 4-{8-amino-3-[(2S)-1-(but-2-ynoyl) pyrrolidin-2-yl]imidazo[1,5-a]pyrazin-1-yl)}-N-(pyridine-2-yl)benzamide.

Acalabrutinib maleate is approved in the form of tablets and marketed by AstraZeneca under the brand name CALQUENCE®. The approved tablets contain Acalabrutinib maleate monohydrate equivalent to 100 mg of acalabrutinib free base.

Acalabrutinib tablets are indicated for the treatment of Mantle Cell Lymphoma, Chronic Lymphocytic Leukemia and Small Lymphocytic Lymphoma.

Acalabrutinib is a small-molecule inhibitor of BTK. Acalabrutinib and its active metabolite, ACP-5862, form a covalent bond with a cysteine residue in the BTK active site, leading to inhibition of BTK enzymatic activity.

CALQUENCE® (Acalabrutinib) tablets for oral administration contains 100 mg acalabrutinib (equivalent to 129 mg of acalabrutinib maleate) and the following inactive ingredients: Low-substituted hydroxypropyl cellulose, Mannitol, Microcrystalline cellulose, Sodium stearyl fumarate, The tablet coating contains Copovidone, Ferric oxide yellow, Ferric oxide red, Hypromellose, Medium-chain triglycerides, Polyethylene glycol 3350, Purified water and titanium dioxide.

WO 2021/255246 A1 discloses solid pharmaceutical dosage forms wherein the dosage form comprises: acalabrutinib maleate in an amount from about 15% to about 55% by weight of the dosage form; at least one diluent in an amount from about 10% to about 70% by weight of the dosage form; at least one disintegrant in an amount from about 0.5% to about 15% by weight of the dosage form; and at least one lubricant in an amount from about 0.25% to about 4% by weight of the dosage form; and wherein the sum of the individual amounts equals 100% of the total weight of the dosage form.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. The above prior art reference discloses solid dosage form comprising specific percentages of Acalabrutinib maleate, diluent, disintegrant and a lubricant. Still, there exists a need to develop an alternative solid dosage form comprising Acalabrutinib maleate which have comparable dissolution properties, content uniformity, stability and equivalent bioavailability w.r.t commercialized Acalabrutinib tablet dosage form.

### Objective of the Invention

The main objective of the present invention relates to a pharmaceutical composition comprising Acalabrutinib maleate.

The present invention also relates to a tablet composition comprising Acalabrutinib maleate and one or more pharmaceutically acceptable excipients.

The present invention also relates to a process for the preparation of tablet composition comprising Acalabrutinib maleate and one or more pharmaceutically acceptable excipients having comparable dissolution properties, content uniformity, stability and equivalent bioavailability w.r.t commercialized Acalabrutinib tablet dosage form.

### SUMMARY OF THE INVENTION

Accordingly, the present invention provides a pharmaceutical composition comprising Acalabrutinib maleate and one or more pharmaceutically acceptable excipients.

The present invention also relates to a pharmaceutical composition comprising Acalabrutinib maleate and one or more pharmaceutically acceptable excipients, wherein the composition is free of a disintegrant.

The present invention also relates to a tablet composition comprising Acalabrutinib maleate and one or more pharmaceutically acceptable excipients.

The present invention also relates to a tablet composition comprising Acalabrutinib maleate and one or more pharmaceutically acceptable excipients, wherein the composition is free of a disintegrant.

The present invention also relates to a process for the preparation of tablet composition comprising Acalabrutinib maleate and one or more pharmaceutically acceptable excipients.

The present invention also relates to a process for the preparation of tablet composition comprising Acalabrutinib maleate and one or more pharmaceutically acceptable excipients, wherein the composition is free of a disintegrant.

### DETAILED DESCRIPTION OF THE INVENTION

The following is a detailed description of embodiments of the present invention. The embodiments are in such detail as to clearly communicate the invention. However, the amount of detail offered is not intended to limit the anticipated variations of embodiments; on the contrary, the intention is to cover all modifications, equivalents, and alternatives falling within the spirit and scope of the present invention as defined by the appended claims.

Unless the context requires otherwise, throughout the specification which follow, the word “comprise” and variations thereof, such as, “comprises” and “comprising” are to be construed in an open, inclusive sense that is as “including, but not limited to.” It is to be appreciated that the terms “comprising”, “comprises” and “comprised of” as used herein includes the terms “consisting of”, “consists” and “consists of” within their meaning.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

The headings and abstract of the invention provided herein are for convenience only and do not interpret the scope or meaning of the embodiments.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Various terms as used herein are shown below. To the extent a term used in a claim is not defined below, it should be given the broadest definition persons in the pertinent art have given that term as reflected in printed publications and issued patents at the time of filing.

The term “active ingredient” or “active agent” or “drug” used interchangeably, is defined to mean active drug, that induce a desired pharmacological or physiological effect.

The term “pharmaceutically acceptable” as used herein means that which is useful in preparing a pharmaceutical composition that is generally safe and non-toxic.

By the term “solid dosage form” or “dosage form” or “composition” as used herein refers to a solid dosage form suitable for administration, such as a tablet, capsule, spheroids, mini-tablets, pellets, granules, pills and the like.

As used in the specification and the appended claims, the singular forms “a”, “an”, and “the” include plural references unless the context clearly dictates otherwise. Thus for example, reference to “a method” includes one or more methods, and/or steps of the type described herein and/or which will become apparent to those persons skilled in the art upon reading this disclosure so forth.

The term “including” is used to mean “including but not limited to”. “Including” and “including but not limited to” are used interchangeably.

The term “excipient” as used herein refers to a components that can be added in the composition, that are physiologically tolerable and do not typically produce allergic or similar untoward reaction. The excipient also provides improved stability and therapeutic enhancement to the composition. The excipients added to the composition to facilitate manufacture, enhance stability, control release, enhance product characteristics, enhance bioavailability, enhance patient acceptability, etc. The term “excipient” and “pharmaceutically acceptable excipient” shall be used interchangeably.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight percentage range of 0.001 to 50% (w/w) should be interpreted to include not only the explicitly recited limits of 0.01% (w/w) to 45% (w/w) but also to include sub-ranges, such as 0.05% to 40% (w/w), 0.1% to 7% (w/w) and so forth, as well as individual amounts, including fractional amounts, within the specified ranges, such as 0.022%, 0.01%, 0.58%, 0.9%, 1.39%, 5.4%, 22.6% for example.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

In another embodiment, the present invention also relates to a pharmaceutical composition comprising Acalabrutinib maleate and one or more pharmaceutically acceptable excipients, wherein the composition is free of a disintegrant.

In one embodiment, the present invention further relates to a tablet composition comprising Acalabrutinib maleate and one or more pharmaceutically acceptable excipients.

In another embodiment, the present invention also relates to a process for preparing a tablet composition comprising Acalabrutinib maleate and one or more pharmaceutically acceptable excipients.

In another embodiment, the present invention also relates to a tablet composition comprising Acalabrutinib maleate and one or more pharmaceutically acceptable excipients, wherein the composition is free of a disintegrant.

In another embodiment, the present invention also relates to a process for the preparation of tablet composition comprising Acalabrutinib maleate and one or more pharmaceutically acceptable excipients, wherein the composition is free of a disintegrant.

In another embodiment, the tablet composition according to the present invention is an immediate release tablet composition.

In one embodiment of the present invention, Acalabrutinib maleate may be present in crystalline form or amorphous form.

In another embodiment of the present invention, Acalabrutinib maleate may be present in crystalline form.

As used herein, the term “% w/w” refers to the weight of the component based on the total weight of a composition comprising the component.

In another embodiment of the present invention, the composition comprises Acalabrutinib maleate in an amount of 20-80% w/w, preferably 30-60% w/w and most preferably 30-50% w/w of the composition.

In another embodiment of the present invention, the composition comprises Acalabrutinib maleate in an amount of 20-80% w/w, preferably 30-60% w/w and most preferably 30-50% w/w of the composition. “Pharmaceutically acceptable excipient/s” are the components added to pharmaceutical formulation to facilitate manufacture, enhance stability, control release, enhance product characteristics, enhance bioavailability, enhance patient acceptability, etc.

In another embodiment, the composition according to the present invention further comprises one or more pharmaceutically acceptable excipients which include but not limited to fillers/diluents, binders, surfactants, glidants and lubricants. These excipients may be present intragranularly or extragranularly.

In another embodiment, the composition according to the present invention is completely “free of a disintegrant” and the pharmaceutically acceptable excipient does not include a disintegrant.

It is to be understood herein that the expression “free of a disintegrant” is meant to characterize the pharmaceutical composition as not comprising a disintegrant type substance(s) or compound(s). On the other hand, it is to be understood herein, that the above expression with respect to the absence of a disintegrant, do not necessarily exclude a substance(s) or compound(s) having a (known) disintegrant function but which may also be exploited so as to effectively take advantage of a different excipient function (e.g. as a binder, diluent, etc.) i.e. in other words such a dual purpose substance(s) or compound(s) may yet be present so as to effectively take advantage of such different excipient function (e.g. as a binder element, diluent element, etc.).

Fillers/diluents according to the present invention include but not limited to microcrystalline cellulose, silicified microcrystalline cellulose, lactose monohydrate, lactose anhydrous, fructose, maltose, trehalose, dextrose, polydextrose, dextrates, dextrins, isomalt, mannitol, maltitol, xylitol, maltodextrin, lactitol, sorbitol, erythritol, inulin, starch, pregelatinized starch, partially pregelatinized starch, sucrose, calcium carbonate, dibasic calcium phosphate, tribasic calcium phosphate, calcium sulfate, magnesium carbonate, magnesium oxide, sodium bicarbonate, sodium carbonate, sodium chloride, cellulose acetate, ethyl cellulose, cellulose powdered, kaolin and the like or combinations thereof. The fillers/diluents can be used in the range of about 0-90% w/w, preferably 10-80% w/w and more preferably 30-60% w/w of the composition.

Binders according to the present invention include but not limited to hydroxypropyl methylcellulose (Hypromellose), hydroxypropyl cellulose, gelatin, ethyl cellulose, polyvinyl alcohol, pregelatinized starch, partially pregelatinized starch, carboxymethyl cellulose, sodium alginate, polyvinyl pyrrolidones (povidone), copovidone, microcrystalline cellulose, gelatin, polymethacrylates, polyethylene glycols (PEG), Poly(vinyl caprolactam-co-vinyl acetate-ethylene glycol) graft polymer (SOLUPLUS®), poloxamers, polyethylene oxide, acrylate based copolymers and the like or combinations thereof. The binder can be used in the range of about 0-15% w/w of the composition.

Surfactants according to the present invention may be selected from anionic, cationic or non-ionic surface-active agents or surfactants. Suitable anionic surfactants include but not limited to carboxylate, sulfonate, and sulfate ions such as sodium lauryl sulfate (SLS), sodium laurate, dialkyl sodium sulfosuccinates particularly bis-(2-ethylhexyl) sodium sulfosuccinate, sodium stearate, potassium stearate, sodium oleate and the like. Suitable cationic surfactants include but not limited to those containing long chain cations, such as benzalkonium chloride, bis-2-hydroxyethyl oleyl amine or the like. Suitable non-ionic surfactants include but not limited to polyoxyethylene sorbitan fatty acid esters (polysorbates), fatty alcohols such as lauryl, cetyl and stearyl alcohols; glyceryl esters such as the naturally occurring mono-, di-, and tri-glycerides; fatty acid esters of fatty alcohols; polyglycolized glycerides such as gelucire; polyoxyethylene-polyoxypropylene block co-polymer such as Poloxamer and other alcohols such as propylene glycol, polyethylene glycol. The surfactant can be used in the range of about 0-20% w/w of the composition.

Glidants according to the present invention include but not limited to silica, colloidal silicon dioxide, talc and magnesium silicate and mixtures thereof. The glidants can be used in the range of 0-10% w/w, preferably 0.5-5% w/w of the composition.

Lubricants according to the present invention include but not limited to stearic acid, magnesium stearate, calcium stearate, sodium stearyl fumarate, talc, zinc stearate, glyceryl mono fatty acid, glyceryl monostearate, glyceryl dibehenate, glyceryl palmito stearic ester, hydrogenated castor oil and mixtures thereof. The Lubricants can be used in the range of 0-10%/o w/w, preferably 0.5-5% w/w of the composition.

In another embodiment, the present invention relates to a tablet composition comprising:
(i) Acalabrutinib maleate,
(ii) one or more diluents,
(iii) one or more lubricants,
(iv) optionally one or more binders,
(v) optionally one or more surfactants, and
wherein the tablet composition is free of a disintegrant.

In another embodiment, the present invention relates to a tablet composition comprising:
(i) Acalabrutinib maleate
(ii) one or more diluents,
(iii) one or more lubricants,
(iv) optionally one or more binders, and
wherein the tablet composition is free of a disintegrant.

In another embodiment, the present invention relates to a tablet composition comprising:
(i) 30-60% w/w of Acalabrutinib maleate,
(ii) 30-60% w/w of one or more diluents,
(iii) 0.5-5% w/w of one or more lubricants, and
(iv) 0-15% w/w of one or more binders,
wherein the tablet composition is free of a disintegrant.

In another embodiment, the present invention relates to a tablet composition,
wherein the tablet composition is further coated with a film coating composition.

In another embodiment, the present invention relates to a tablet composition comprising:
(i) Acalabrutinib maleate,
(ii) one or more diluents,
(iii) one or more lubricants,
(iv) optionally one or more binders,
(v) optionally one or more surfactants,
(vi) a film coating over the tablet composition, and
wherein the tablet composition is free of a disintegrant.

In another embodiment, the present invention relates to a tablet composition comprising:
(i) Acalabrutinib maleate,
(ii) one or more diluents,
(iii) one or more lubricants,
(iv) optionally one or more binders,
(vi) a film coating over the tablet composition, and
wherein the tablet composition is free of a disintegrant. In another embodiment, the present invention relates to a tablet composition consisting of:
(i) 30-60% w/w of Acalabrutinib maleate,
(ii) 30-60% w/w of a diluent, and
(iii) 0.5-5% w/w of a lubricant,
wherein the tablet composition is free of a disintegrant.

In another embodiment, the present invention relates to a tablet composition consisting of:
(i) 30-60% w/w of Acalabrutinib maleate,
(ii) 30-60% w/w of a diluent,
(iii) 0.5-5% w/w of a lubricant, and
(iv) a film coating over the tablet composition,
wherein the tablet composition is free of a disintegrant.

In another embodiment, the present invention relates to a tablet composition, wherein:
one or more diluents are selected from pregelatinized starch, mannitol, microcrystalline cellulose, silicified microcrystalline cellulose and lactose;
one or more lubricants are selected from sodium stearyl fumarate and magnesium stearate; and
one or more binders are selected from povidone, hydroxypropyl cellulose and hydroxypropyl methylcellulose.

In another embodiment, the present invention relates to a tablet composition comprising:
(i) 30-60% w/w of Acalabrutinib maleate,
(ii) 30-60% w/w of one or more diluents selected from pregelatinized starch, mannitol, microcrystalline cellulose, silicified microcrystalline cellulose and lactose,
(iii) 0.5-5% w/w of one or more lubricants selected from sodium stearyl fumarate and magnesium stearate, and
(iv) 0-15% w/w of one or more binders selected from povidone, hydroxypropyl cellulose and hydroxypropyl methylcellulose.

In another embodiment, the present invention relates to a tablet composition consisting of;
(i) 30-60% w/w of Acalabrutinib maleate
(ii) 30-60% w/w of pregelatinized starch,
(iii) 0.5-5% w/w of sodium stearyl fumarate, and
(iv) a film coating over the tablet composition,
wherein the tablet composition is free of a disintegrant.

In another embodiment, the present invention relates to a process for the preparation of pharmaceutical composition, comprising the steps of:
(i) blending Acalabrutinib maleate with one or more pharmaceutically acceptable excipients,
(ii) formulating the blend of step (i) into suitable dosage form.

In another embodiment, the present invention relates to a process for the preparation of pharmaceutical composition, comprising the steps of:
(i) blending Acalabrutinib maleate with one or more pharmaceutically acceptable excipients,
(ii) optionally, lubricating the blended material of step (i) with a lubricant, and
(iii) preparing the lubricated material of step (ii) into a suitable dosage form.

In another embodiment, the present invention relates to a process for the preparation of pharmaceutical composition, comprising the steps of:
(i) blending Acalabrutinib maleate with one or more pharmaceutically acceptable excipients,
(ii) optionally, lubricating the blended material of step (i) with a lubricant, and
(iii) compressing the lubricated material of step (ii) into tablets.

In another embodiment, the present invention relates to a process for the preparation of pharmaceutical composition, comprising the steps of:
(i) blending Acalabrutinib maleate with one or more pharmaceutically acceptable excipients,
(ii) optionally, lubricating the blended material of step (i) with a lubricant, and
(iii) compressing the lubricated material of step (ii) into tablets, and
(iv) optionally coating the tablets obtained in step (iii) with a film coating composition.

In another embodiment, the present invention relates to a process for the preparation of pharmaceutical composition, comprising the steps of:
(i) blending Acalabrutinib maleate with one or more pharmaceutically acceptable excipients,
(ii) optionally, lubricating the blended material of step (i) with a lubricant, and
(iii) compressing the lubricated material of step (ii) into tablets, and
(iv) coating the tablets obtained in step (iii) with a film coating composition.

In another embodiment, the present invention relates to a process for the preparation of pharmaceutical composition, comprising the steps of:
(i) blending Acalabrutinib maleate with one or more pharmaceutically acceptable excipients,
(ii) granulating the blend of step (i),
(iii) blending the granules of step (ii) with one or more pharmaceutically acceptable excipients, and
(iv) compressing the blend of step (iii) into tablets.

In another embodiment, the present invention relates to a process for the preparation of pharmaceutical composition, comprising the steps of:
(i) blending Acalabrutinib maleate with one or more pharmaceutically acceptable excipients,
(ii) granulating the blend of step (i),
(iii) blending the granules of step (ii) with one or more pharmaceutically acceptable excipients,
(iv) compressing the blend of step (iii) into tablets, and
(v) optionally coating the tablets obtained in step (iv) with a film coating composition.

In another embodiment, the present invention relates to a process for the preparation of pharmaceutical composition, comprising the steps of:
(i) blending Acalabrutinib maleate with one or more pharmaceutically acceptable excipients,
(ii) granulating the blend of step (i),
(iii) blending the granules of step (ii) with one or more pharmaceutically acceptable excipients,
(iv) compressing the blend of step (iii) into tablets, and
(v) coating the tablets obtained in step (iv) with a film coating composition.

In another embodiment, the present invention relates to a process for the preparation of tablet composition, comprising the steps of:
(i) blending Acalabrutinib maleate with one or more diluents,
(ii) granulating the blend of step (i),
(iii) blending the granules of step (ii) with one or more lubricants,
(iv) compressing the blend of step (iii) into tablets, and
(v) coating the tablets obtained in step (iv) with a film coating composition.

In another embodiment, the present invention relates to a process for the preparation of tablet composition, comprising the steps of:
(i) blending Acalabrutinib maleate with one or more diluents,
(ii) lubricating the blend of step (i) with one or more lubricants,
(iii) compressing the blend of step (ii) into tablets, and
(iv) coating the tablets obtained in step (iii) with a film coating composition.

In another embodiment, the present invention relates to a process for the preparation of tablet composition, comprising the steps of:
(i) blending Acalabrutinib maleate with pregelatinized starch,
(ii) granulating the blend of step (i) using roller compaction,
(iii) lubricating the granules of step (ii) with sodium stearyl fumarate,
(iv) compressing the lubricated blend of step (iii) into tablets, and
(v) coating the tablets obtained in step (iv) with a film coating composition.

In another embodiment, the present invention relates to a process for the preparation of pharmaceutical composition, comprising the steps of;
(i) blending Acalabrutinib maleate with one or more pharmaceutically acceptable excipients,
(iii) filling the blend of step (i) into capsules.

In another embodiment, the present invention relates to a process for the preparation of pharmaceutical composition, comprising the steps of:
(i) blending Acalabrutinib maleate with one or more pharmaceutically acceptable excipients,
(ii) lubricating the blended material of step (i) with a lubricant, and
(iii) filling the lubricated material of step (ii) into capsules.

In another embodiment, the present invention relates to a process for the preparation of pharmaceutical composition, comprising the steps of:
(i) blending Acalabrutinib maleate with one or more pharmaceutically acceptable excipients,
(ii) granulating the blend of step (i),
(iii) blending the granules of step (ii) with one or more pharmaceutically acceptable excipients, and
(iii) filling the blend of step (iii) into capsules.

In another embodiment, the pharmaceutical composition according to the present invention is in the form of tablets, capsules, granules, powder, pellets and sachets.

In another preferred embodiment, the pharmaceutical composition according to the present invention is in the form of tablets.

In another embodiment, the blend is formulated into a suitable dosage form like tablets or capsules using different techniques which are well known in the prior art.

In another embodiment, the compositions of the present invention may be prepared using any method known in the art, but are not limited to wet granulation, melt granulation, dry granulation, roller compaction, solid dispersion, encapsulation and direct compression. In another embodiment, the granulation can be done using one pharmaceutically acceptable excipient, a binder, which can be added to the drug substance in a dissolved state (e.g. in an aqueous/non-aqueous solution) or in a powder form and then granulated by adding a granulation liquid. A combination of more than one binder can be used.

In another embodiment, the solvents used for granulation process may be selected from water, isopropyl alcohol, methanol, ethanol, methylene chloride or combination thereof.

In another embodiment, the granulation can be done using any method known in the art, but are not limited to fluidized bed granulation, high-shear granulation, low shear granulation, spray granulation, melt granulation and hot melt extrusion.

The pharmaceutical composition may be further film coated with functional or non functional layer. The coating may be selected from amongst one or more of those suitable coating materials known in the art. For example, the coating material can be Opadry or Opadry AMB. Coating may be performed by applying one or more film forming polymers, with or without other pharmaceutically inert excipients, as a solution/suspension using any conventional coating technique known in the art, such as spray coating in a conventional coating pan or fluidized bed processor; or dip coating.

Coloring agent may be selected from FDA approved colorants such as Iron Oxide, Lake of Tartrazine, Allura Red, Lake of Quinoline Yellow, Lake of Erythrosine, Titanium Dioxide and the like.

The composition according to the present invention may be uncoated or optionally coated with functional coating, film coating, moisture barrier coating or a protective coating composition. The coating may be selected from amongst one or more of those suitable coating materials known in the art. Coating may be performed by applying one or more film forming polymers, with or without other pharmaceutically inert excipients, as a solution/suspension using any conventional coating technique known in the art, such as spray coating in a conventional coating pan or fluidized bed processor or dip coating.

The amount of the film coating may be about 1 to about 10% w/w, preferably, about 1 to about 3% w/w, of the total composition. Any of a variety of film coatings can be used in the present composition. Suitable film coating may include but not limited to polymers, plasticizers, pigments, opacifiers, glidants, binders, antitacking agents, antifoaming agents, surfactants, fillers, extenders, coloring agents and the like.

Examples of film-forming polymers include ethylcellulose, hydroxypropyl methylcellulose, polyvinyl alcohol (PVA), hydroxypropylcellulose, methylcellulose, carboxymethyl cellulose, hydroxymethylcellulose, hydroxyethylcellulose, cellulose acetate, hydroxypropyl methylcellulose phthalate, cellulose acetate phthalate, cellulose acetate trimellitate; waxes such as polyethylene glycol; methacrylic acid polymers such as Eudragit® RL and RS; and the like. Alternatively, commercially available coating compositions comprising film forming polymers marketed under various trade names, such as Opadry® may also be used for coating the tablets. The coating can be obtained as a dry blend concentrate.

The film coating may also optionally include a plasticizer such as triacetin, propylene glycol, diethyl phthalate, tributyl sebacate or polyethylene glycol (PEG), preferably PEG; and an anti-adherent or glidant such as talc, fumed silica or magnesium stearate, an opacifying agent such as titanium dioxide.

Coloring agent may be selected from FDA approved colorants such as Iron Oxide, Lake of Tartrazine, Allura Red, Lake of Quinoline Yellow, Lake of Erythrosine, titanium dioxide and the like.

The coating according to the present invention is applied by solubilising or suspending the excipients in solvents such as isopropyl alcohol, water, acetone, ethanol, methylene chloride, hydrochloric acid and the like, or mixtures thereof.

In another embodiment, the pharmaceutical composition according to the present invention is in the form of tablets, preferably immediate release tablets.

In another embodiment, the pharmaceutical composition according to the present invention is in the form of tablets which may be coated or uncoated.

In another embodiment, the tablet according to the present invention may be round or oval. The edges of the tablets can be beveled or rounded. In another embodiment, the tablets are ovoid or round. The tablets according to the invention may be scored.

In another embodiment, the present invention provides a tablet composition comprising Acalabrutinib free base (Equivalent to Acalabrutinib maleate) in the range of about 1 mg to about 500 mg, preferably 50 mg to 200 mg and more preferably 100 mg.

In another embodiment, the present invention provides a tablet composition comprising Acalabrutinib maleate for the treatment of patients with Mantle Cell Lymphoma, Chronic Lymphocytic Leukemia and Small Lymphocytic Lymphoma.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

EXAMPLE 1
Tablet composition comprising Acalabrutinib maleate

| | | Quantity | |
|---|---|---|---|
| S. No | Ingredients | (mg/tab) | % w/w |
| 1 | Acalabrutinib maleate | 129.00 | 48.68 |
| 2 | Silicified Microcrystalline cellulose | 129.00 | 48.68 |
| 3 | Sodium stearyl fumarate | 7.00 | 2.64 |
| | Total Tablet Weight | 265.00 | 100.00 |

The processing steps involved in manufacturing the tablets were given below:
(i) Acalabrutinib maleate and Silicified Microcrystalline cellulose were sifted separately and blended,
(ii) the blend of step (i) was lubricated with sodium stearyl fumarate stearate, and
(iii) the lubricated granules of step (ii) were compressed into tablets.

EXAMPLE 2
Tablet composition comprising Acalabrutinib maleate

| | | Quantity | |
|---|---|---|---|
| S. No | Ingredients | (mg/tab) | % w/w |
| | Intra-granular | | |
| 1 | Acalabrutinib maleate | 129.00 | 48.68 |
| 2 | Lactose monohydrate | 124.00 | 46.79 |
| 3 | Povidone | 7.00 | 2.64 |
| 4 | Purified water | q.s | |
| | Extra-granular | | |
| 5 | Sodium stearyl fumarate | 5.00 | 1.88 |
| | Total Tablet Weight | 265.00 | 100.00 |

The processing steps involved in manufacturing the tablets were given below.
(i) Acalabrutinib maleate and lactose monohydrate were sifted separately and blended,
(ii) Povidone was dissolved in purified water to prepare binder solution,
(iii) the blend of step (i) was granulated using the binder solution of step (ii), milled and dried,
(iv) the granules obtained in step (iii) was lubricated with sodium stearyl fumarate, and
(v) the lubricated granules of step (iv) were compressed into tablets.

EXAMPLE 3
Tablet composition comprising Acalabrutinib maleate

| | | Quantity | |
|---|---|---|---|
| S. No | Ingredients | (mg/tab) | % w/w |
| | Intra-granular | | |
| 1 | Acalabrutinib maleate | 129.00 | 48.68 |
| 2 | Mannitol | 129.00 | 48.68 |
| | Extra-granular | | |
| 3 | Sodium stearyl fumarate | 7.00 | 2.64 |
| | Total Tablet Weight | 265.00 | 100.00 |

The processing steps involved in manufacturing the tablets were given below.
(i) Acalabrutinib maleate and mannitol were sifted separately and blended,
(ii) the blend of step (i) was compacted using roller compaction and milled,
(iii) the milled granules obtained in step (ii) were lubricated with sodium stearyl fumarate, and
(iv) the lubricated granules of step (iii) were compressed into tablets.

EXAMPLE 4
Tablet composition comprising Acalabrutinib maleate

| | | Quantity | |
|---|---|---|---|
| S. No | Ingredients | (mg/tab) | % w/w |
| | Intra-granular | | |
| 1 | Acalabrutinib maleate | 129.00 | 48.68 |
| 2 | Pregelatinized Starch | 129.00 | 48.68 |
| | Extra-granular | | |
| 3 | Sodium stearyl fumarate | 7.00 | 2.64 |
| | Total Tablet Weight | 265.00 | 100.00 |
| | Film Coating | | |
| | Opadry | 10.00 | |
| | Total Film coated Tablet weight | 275.00 | |

The processing steps involved in manufacturing the tablets were given below:
(i) Acalabrutinib maleate and Pregelatinized Starch were sifted separately and blended,
(ii) the blend of step (i) was compacted using roller compaction and milled,
(iii) the milled granules obtained in step (ii) were lubricated with sodium stearyl fumarate,
(iv) the lubricated granules of step (iii) were compressed into tablets, and
(v) the tablets of step (iv) were coated with OPADRY coating solution.
   Dissolution Data: Table 1 given below provides the comparative dissolution profile of Acalabrutinib maleate 100 mg tablets prepared according to Example 4 with CALQUENCE® 100 mg tablets carried out in 500 ml of 0.1N HCl as dissolution medium in USP II apparatus (paddle) at 50 rpm.

TABLE 1
Comparative dissolution profile of Acalabrutinib maleate
100 mg tablets prepared according to Example 4 with
CALQUENCE ® 100 mg tablets

Cumulative % drug released

| | CALQUENCE ® | Acalabrutinib maleate |
|---|---|---|
| | (Acalabrutinib | 100 mg tablets |
| Time | maleate) 100 mg | prepared according |
| (in min) | tablets | to Example 4 |
| 5 | 83.0 | 93.4 |
| 10 | 92.4 | 97.9 |
| 15 | 94.6 | 98.6 |
| 20 | 96.5 | 99.2 |
| 30 | 99.6 | 99.8 |

Based on the above data more than 94% release was observed within 15 minutes in both Acalabrutinib maleate 100 mg tablets prepared according to Example 4 and CALQUENCE (Acalabrutinib maleate) 100 mg tablets. Hence, it was concluded that the dissolution profile of Acalabrutinib maleate 100 mg tablets prepared according to Example 4 was comparable with CALQUENCE® (Acalabrutinib maleate) 100 mg tablets.
The Acalabrutinib maleate 100 mg tablets prepared according to Example 4 despite of being free from disintegrating agent exhibited comparable dissolution profile in comparison to reference CALQUENCE® (Acalabrutinib maleate) 100 mg tablets which contain a disintegrating agent.

### Stability Data:

Table 2 given below shows the impurity profile of Acalabrutinib maleate 100 mg tablets prepared according to Example 4 of the present invention after storing at 40 f 2 °C/75±5% RH for 2 months.

TABLE 2
Stability data of Acalabrutinib maleate 100 mg
tablets prepared according to Example 4 after
storing at 40 + 2 °C/75 + 5% RH for 2 months.

| Characteristics | Initial | 1^{st} month | 2nd month |
|---|---|---|---|
| Assay | 101.4% | 99.6% | 99.9% |
| Moisture content | 3.9% | 2.3% | 2.9% |
| Total Impurities | 0.11% | 0.12% | 0.13% |

The above stability study results of the Acalabrutinib maleate 100 mg tablets prepared according to Example 4 of the present invention indicates that, the tablets remain stable for a period of 2 months even after storing at 40±2 °C/75±5% RH. Further, the total impurities were found to be within the specified limits.

We claim:

1. A tablet composition comprising:
(i) 30-60% w/w of Acalabrutinib maleate,
(ii) 30-600% w/w of one or more diluents,
(iii) 0.5-5% w/w of one or more lubricants, and
(iv) 0-15% w/w of one or more binders,
wherein the tablet composition is free of a disintegrant.

2. The tablet composition as claimed in claim 1, wherein the tablet composition is further coated with a film coating composition.

3. The tablet composition as claimed in claim 1, wherein one or more diluents are selected from pregelatinized starch, mannitol, microcrystalline cellulose, silicified microcrystalline cellulose and lactose.

4. The tablet composition as claimed in claim 1, wherein the tablet composition comprises:
(i) 30-60% w/w of Acalabrutinib maleate,
(ii) 30-60% w/w of one or more diluents selected from pregelatinized starch, mannitol, microcrystalline cellulose, silicified microcrystalline cellulose and lactose,
(iii) 0.5-5% w/w of one or more lubricants selected from sodium stearyl fumarate and magnesium stearate, and
(iv) 0-15% w/w of one or more binders selected from povidone, hydroxypropyl cellulose and hydroxypropyl methylcellulose.

5. The tablet composition as claimed in claim 1, wherein the tablet composition is prepared by dry granulation, roller compaction, wet granulation, melt granulation, and direct compression.

6. The tablet composition as claimed in claim 1, wherein the tablet composition is prepared by a process comprising the steps of:
(i) blending Acalabrutinib maleate with one or more diluents,
(ii) granulating the blend of step (i),
(iii) blending the granules of step (ii) with one or more lubricants,
(iv) compressing the blend of step (iii) into tablets, and
(v) coating the tablets obtained in step (iv) with a film coating composition.

7. The tablet composition as claimed in claim 1, wherein the tablet composition is prepared by a process comprising the steps of:
(i) blending Acalabrutinib maleate with one or more diluents,
(ii) lubricating the blend of step (i) with one or more lubricants,
(iii) compressing the blend of step (ii) into tablets, and
(iv) coating the tablets obtained in step (iii) with a film coating composition.

8. A tablet composition consisting of:
(i) 30-60% w/w of Acalabrutinib maleate
(ii) 30-60% w/w of a diluent, and
(iii) 0.5-5% w/w of a lubricant,
wherein the tablet composition is free of a disintegrant.

9. A tablet composition consisting of:
(i) 30-60% w/w of Acalabrutinib maleate
(ii) 30-60% w/w of pregelatinized starch,
(iii) 0.5-5% w/w of sodium stearyl fumarate, and
(iv) a film coating over the tablet composition,
wherein the tablet composition is free of a disintegrant.

10. The tablet composition as claimed in claim 1, wherein the tablet composition is prepared by a process comprising the steps of:
(i) blending Acalabrutinib maleate with pregelatinized starch,
(ii) granulating the blend of step (i) using roller compaction,
(iii) lubricating the granules of step (ii) with sodium stearyl fumarate,
(iv) compressing the lubricated blend of step (iii) into tablets, and
(v) coating the tablets obtained in step (iv) with a film coating composition.
